(19) United States
(12) Patent Application Publication
(10) Pub. No.: US 2024/0416121 A1
(43) Pub. Date: Dec. 19, 2024

(54) SYSTEM FOR ACTIVATING NERVE CELLS IN THE HUMAN EYE AND BRAIN

(71) Applicant: SAVIR GMBH, Berlin (DE)

(72) Inventor: Kornelia Sabel, Berlin (DE)

(21) Appl. No.: 18/701,870

(22) PCT Filed: Feb. 16, 2022
(86) PCT No.: PCT/EP2022/053801

§ 371 Date: Apr. 16, 2024

(30) Foreign Application Priority Data
Nov. 24, 2021 (DE) 10 2021 130 837.2
Dec. 14, 2021 (DE) 10 2021 133 100.5

(51) Int. Cl. A61N 1/36; A61M 21/00; A61M 21/02; A61N 1/04; A61N 5/06

(52) U.S. Cl. CPC A61N 1/36046; A61M 21/02; A61N 1/0456; A61N 5/0625; A61M 2021/0027; A61M 2021/0066; A61M 2021/0072; A61N 2005/0659

(57) ABSTRACT

The invention relates to a system for activating nerve cells in the human eye and brain for enhancing the residual vision in existing visual-field defects, for reducing or correcting other visual disturbances or cognitive deficits by means of neurostimulation, in particular non-invasive application of microcurrent to the head, more particularly non-invasive transcranial alternating current stimulation (tACS), the system consisting of: a non-invasive applicator for guiding a flow of energy to the eye and brain and for stimulating the blood flow and activation of nerve cells, in particular retinal ganglion cells and brain cells; an impulse generator for generating electrical or magnetic stimulation signals; and a data-processing and control unit for providing patient-specific stimulation signal sequences; wherein the applicator comprises for example at least two electrodes, which can be brought into contact with the head of the test subject and in this respect stimulation electrodes, in particular replaceable stimulation electrodes, are fixed on the applicator such that they rest or make contact on the right and left of the eye in the region of the temple of the patient's head; wherein the stimulation signal sequences are provided repeatedly for application until a demonstrable reduction in an existing vascular dysregulation in the eyes and in the brain is determined.

The invention relates to a system for activating nerve cells in the human eye and brain for enhancing residual vision in the case of existing visual-field defects, for reducing or correcting other visual disorders or cognitive deficits by means of neuromodulation, in particular non-invasive microcurrent application to the head, more particularly non-invasive transcranial alternating current stimulation (tACS), consisting of an applicator for guiding an energy flow to the eye and brain and for stimulating blood flow and activation of nerve cells, in particular retinal ganglion cells, an impulse generator for generating electrical or magnetic stimulation signals and a data-processing and control unit for providing patient-specific stimulation signal sequences, wherein the applicator comprises, for example, at least two electrodes which can be brought into contact with the head of the test subject and in this respect stimulation electrodes, in particular replaceable electrodes, are fixed to the applicator in such a way that they preferably rest on or against the right and left of the eye in the region of the temple of the patient's head, according to claim 1.

Visual-field defects have so far been considered irreversible, as nerve cells in the retina, optic nerve and brain cannot regenerate. However, it has been shown that a partial recovery of visual-field defects is possible, as the brain, which evaluates and interprets retinal signals, can permanently amplify the residual signals via certain mechanisms. There are also nerve cells in the retina that reduce their function after a pathological event and “die”, as they can no longer send out nerve signals without dying.

As experience has shown that almost all patients still have residual vision, i.e. so-called residual vision, it has been proposed to reactivate the “silent” nerve cells and to strengthen residual vision by improving synaptic transmission. Clinical studies show the success of treatment methods such as visual training or alternating current stimulation. In alternating current treatment, the entire retina and parts of the brain are activated and synchronized. The vast majority of patients respond well to the above-mentioned forms of therapy. Physiological studies of the mechanisms of alternating current with EEG and fMRI indicate a massive local and global change in blood flow and the reorganization of neuronal networks in the brain. A local activation of nerve cells takes place in the eyes and in the visual system of the brain via a global reorganization of neuronal networks. As residual vision can be strengthened by reactivating nerve cells and modulating neuroplasticity, not only the eyes but also the brain are of great importance for visual rehabilitation in ophthalmology.

For some time now, the adaptability of the brain, also known as plasticity, has been used in ophthalmology. Using methods of neuromodulation by means of weak alternating currents, partially damaged visual functions can be strengthened via the process of neuroplasticity.

Recently, methods to improve blood flow and resynchronize the brain's network activity using alternating current pulses have been tested on partially blind patients. Here, alternating current is administered non-invasively for several days in defined sessions using special electrodes on the forehead. The alternating current flows from the electrodes to the eye, where it stimulates retinal ganglion cells to fire at predetermined frequencies with the aim of resynchronizing brain networks. Damage to the optic nerve leads to a disorganization of functional networks in the brain, which is reflected by a neurophysiological desynchronization in the EEG. Treatments using alternating current can significantly improve this condition by improving blood circulation and resynchronizing brain network activity.

However, the resynchronization of visual performance varies greatly from person to person and depends on a wide range of factors.

In addition to optimizing the stimulation impulses, it is also important to keep the patient largely stress-free, at least during treatment. For this purpose, it is necessary to create an optimized design of stimulation electrodes, including technical means for holding these electrodes on the patient's head. An applicator must therefore be created that is both ergonomically optimal and particularly suitable for treatment and meets all requirements.

It is also essential to optimize the stimulation in such a way that treatment success is maintained beyond the stimulation period and for as long as possible.

With regard to the prior art, reference is made to EP 1 603 633 B1, which shows a special attachment part for a stimulation device at a desired position on the patient's head, wherein an adjustment structure is made of a deformable material which is capable of adapting the device to the contours of the patient's head.

Fastening devices or arrangements for contacting stimulation electrodes are also disclosed in EP 1 708 787 and EP 1 734 877 B1.

With regard to the prior art in brain stimulation with additional control of the blood flow behavior of neighboring vessels, attention is drawn to WO 2011/106660 A1 and WO 2016/115392 A1.

WO 2011/106660 A1 focuses on recognizing the effect of stimulation and, in particular, the simultaneous stimulation and recording of the stimulation effect in real time. For this purpose, functional near-infrared spectroscopy is used in a non-invasive manner during stimulation. The functional near-infrared spectroscopy is used to measure the oxygenation status of hemoglobin.

WO 216/115392 A1 relates to devices and methods for determining neurovascular reactivity using stimulation of the neurovascular system and simultaneous recording of a neuronal hemodynamic response thereto.

With regard to the various possibilities for forming stimulation electrodes and corresponding applicators, attention is drawn to the prior art briefly outlined below.

U.S. Pat. No. 5,522,864 A discloses a headband-like electrode holder. A first electrode is applied to the closed eyelid of a patient. The second electrode is fixed in the headband. A third electrode is attached in the neck area. This type of electrode formation, in particular the attachment of an electrode to a closed eyelid, is perceived as unpleasant by the patient and puts him under negative stress for the Success of the treatment.

Also in the patent family around EP 30 13 414 B1, which shows a stimulation device for transdermal electrical stimulation, several electrodes are fixed at positions clearly distant from each other. A first electrode is attached to the forehead area of the patient. A second electrode is located on the patient's neck or shoulder. This electrode arrangement is difficult to individualize and can only be optimally positioned by the patient with the help of a third party, which is a considerable disadvantage for home therapy.

EP 33 49 844 A1 deals with treatment using microcurrents. In this regard, punctual stimulation electrodes are applied above and below the eye in the eyelid area. Such positioning directly in the sensitive eye area is uncomfortable for many patients, particularly because the electrodes are relatively small and therefore do not provide sufficient current strength. Even very low currents can be felt, but these are not sufficiently effective therapeutically. Especially in the area above and below the eye, the human skin is very sensitive to irritation, so that this type of electrode design has not been widely used.

EP 2 981 326 B1 shows a head-set-like arrangement for electrostimulation of the skin surface of the head.

DE 10 2011 055 844 B4 discloses a spectacle-like carrier for stimulation electrodes, with an additional nose support similar to typical spectacles. The electrodes are designed to be removable, i.e. replaceable. However, this arrangement uses hair electrodes that lie directly on the cornea of the eyeball, which entails risks of corneal damage.

In EP 2 651 504 B1, a headband is provided as an electrode carrier for neurostimulation, wherein the headband is applied to the head area of the patient and the corresponding electrodes are applied symmetrically to the back of the patient's head. There are also electrical connections for connecting the electrodes to a separate impulse generator.

The device according to US 2006/129207 A for the electrical stimulation of ganglion cells is again based on a spectacle-like structure that has electrodes that are connected to the eyelid of the test person. A support is intended to minimize the otherwise unpleasant pressure on the eyelids, but this results in the disadvantage that the contact resistance between the electrodes and the subject's skin is not always clearly reproducible.

In summary, the prior art shows a large number of different applicators for electrical stimulation, including for local activation of the eyes and brain, wherein the majority of the known applicators are not suitable for home therapy, i.e. in the sense of reproducible, simple and risk-free application by the patient without medical or physician support.

There are currently no known effective treatment measures or systems to treat patients with or after Covid disease or vaccination who suffer from prolonged visual impairment or cognitive deficits such as impaired attention, memory, speech comprehension or fatigue.

It has been recognized that a Covid infection causes circulatory disorders that can lead to neuronal dysfunction.

Currently, a large number of Covid patients report various functional complaints in the sense of a persistent state of illness. This also occurs in non-hospitalized people who initially had only mild Covid-19 symptoms, which later develop into symptoms such as headaches, chest pain, muscle pain, tingling and stinging, loss of smell, persistent cough, shortness of breath, palpitations or diarrhea, as well as abdominal pain, skin rash, fever, fatigue, forgetfulness and depression.

Long-term Covid patients also show abnormalities in mood functions and lower stress resistance.

In addition to cognitive and emotional impairments, long-term Covid patients can also have visual problems. However, these have not yet been recognized by patients or treating physicians, as such problems tend to be benign and are rarely investigated. However, it is clear from publications that Covid patients not only have viral RNA in their eyes and abnormalities of the retinal vessels, but that over 20% of asymptomatic Covid-19 patients have retinal microangiopathy and reduced vascular and blood flow density in the macula. There are also cases of acute macular neuroretinopathy.

A major problem of Covid-19 is the disruption of blood vessel health, which can affect blood flow throughout the body, with very small microvessels with their relatively large vessel wall surface area being most at risk.

Since long-term Covid problems are spreading and no effective treatment for improving visual and cognitive limitations is available so far, it is the object of the invention to provide a system that enables an improvement of the blood flow regulation in the eye, i.e. in the brain, or restores the blood flow regulation, so that ultimately the synchronization of the functional connectivity networks in the brain are improved.

According to the invention, it was recognized that a system which uses neuromodulation with the aid of non-invasive electrical brain stimulation is helpful in this respect.

According to the invention, transorbital alternating current stimulation (tACS) is preferred. Such alternating current stimulation forces the neurons to fire action potentials and improves blood flow. This dual effect leads to a partial restoration of disturbed neuronal networks in the brain, which has a clinically positive effect on vision. The use of the system is able to reduce long-lasting Covid symptoms. Just three to ten days of treatment using the system according to the invention leads to a long-lasting recovery of visual fields and cognitive functions.

The system-organized microcurrent treatment leads to a current flow in the eyes, the optic nerve and the frontal cortex, midbrain and brain stem of the brain.

This current flow improves neuronal synchronization and blood circulation. In the eye and brain, the vascular regulation and oxygen supply to the nerve cells is normalized, which was disturbed by Covid.

The system is used daily over a longer period of time, for example two to fifteen days.

The current application uses alternating or alternatively direct current treatment.

For example, alternating current with a difference of 10 Hertz is used. The current strength is variable and lies, for example, in the range between 200 and 1000 μA.

According to the invention, the current intensity is slowly increased in steps, i.e. ramped up, depending on whether the respective patient perceives visual impressions in the form of flashes of light or the like.

The increment can be 100 μA, for example.

The solution to the problem according to the invention is achieved with a system according to the features of claim 1, wherein the subclaims comprise at least expedient embodiments and further developments.

Thus, according to the invention, the stimulation signal sequences are repeatedly provided for application until a detectable reduction of an existing vascular dysregulation in the eyes and in the brain is determined. Such a determination can be carried out automatically, using a recording of the vascular dynamics in the retina together with the acquisition of the corresponding data. For example, the vascular dynamics in the retina can be determined before and after treatment using a Dynamic Vascular Analyzer (DVA).

The analyzer quantifies the response of retinal vasodilatation by measuring the vessel diameters before, during and after flicker light stimulation. This provides information on the condition of the arterial and venous vessels.

Since the retina is a tissue of the central nervous system, i.e. with brain-like neurons and vessels, such an analysis can be considered a biomarker for the vascular health of the brain.

To measure the vascular dynamics, the retina can be videotaped with a fundus camera at a given field of view.

The fundus camera is then connected to the aforementioned analysis system.

In order to obtain a complete image of the patient's central retina, the pupils are dilated.

In one example, the retina of each eye was imaged during a five- to six-minute session consisting of several measurement periods and stored on video.

For example, a fifty-second baseline measurement, three repeated 10 Hz flicker stimulation period measurements of twenty seconds each and a subsequent eighty-second post-flicker period.

The three twenty-second flicker stimulation periods are averaged. The video is then analyzed for changes in vessel diameter over time to evaluate the dynamics.

In a healthy retina, flickering triggers neuronal activation, which then causes vasodilation due to neurovascular coupling mechanisms.

In addition to the DVA analysis programs used, the absolute vessel diameter is determined to prove the effectiveness of the system.

As a result of the practical application of the system according to the invention, patients reported that their visual field recovered after treatment. A measurement of visual acuity showed improvements in both eyes.

There was also an improvement in almost all cognitive areas after using the system.

All in all, the system according to the invention enables the application of neuromodulation of the eye and the brain in the sense of a short-term treatment to increase attention, improvement in memory performance and a restoration of speech comprehension, especially in patients with Long Covid symptoms.

The use of the system improves blood flow and reduces vascular dysregulation. This results in neuronal synchronization of the brain's functional connectivity networks and a corresponding improvement can be demonstrated. The severity of cognitive symptoms in long-term Covid patients is reduced.

Decisive for the success and acceptance of the system according to the invention and the means available in the system is the use of an intuitively usable applicator, wherein an optimal stimulation signal sequence can be found in combination with an arrangement for the non-invasive determination of the blood flow to the skin and brain of the test person and for the determination of the oxygen saturation, which leads to a maximum increase in blood flow and the longest-lasting after-effect, such that the increase in blood flow and neuronal activation continues for as long as possible after stimulation, which is a clear indicator of the desired success of the treatment.

The impulse generator for generating the stimulation signal sequences can be a single-channel or multi-channel stimulator that interacts with a data processing and control unit so that patient-specific stimulation signal sequences are generated.

The temporal positioning makes it possible to stimulate both eyes simultaneously with only one channel, which reduces the cost of manufacturing the stimulation generator and improves the effectiveness and reliability, i.e. a lower variability of success.

The optimum stimulation signal sequences can be determined during an initial treatment under medical supervision, then saved and transferred to the patient on a patient memory card for home use. It is also possible to store data directly in a memory unit of the applicator.

The applicator is designed so that it can be used intuitively after a brief explanation of how to use it, especially at home.

Since it has been shown that treatment is particularly successful if the patient is kept stress-free immediately before and during the treatment, the applicator in one embodiment of the invention has sound generating means, in particular in the form of earphones or headphones. On the one hand, these sound generating means can be used to inform the patient how to operate the applicator, which program to select, how long the treatment lasts or the like. In addition, voice messages, sound sequences or music can be played to induce a desired relaxation response.

The applicator therefore comprises an electrode holder that can be individually adjusted to the shape of the head. Furthermore, the electrodes are positioned at the temples for optimum stimulation. This has the advantage that the current intensity of the pulse sequences can be reduced while still generating sufficient phosphenes. An NIR feedback system can be used to maximize the hemodynamic after-effect during non-invasive eye and brain simulation.

In the course of extensive investigations and studies, going back to the applicant, it has been shown that the arrangement of the electrodes plays a decisive role in the stimulation success of the visual system.

In this regard, a so-called F7-F8 arrangement according to the nomenclature of the EEG 10-20 system in the area of the patient's temples is proposed. In such an arrangement, phosphenes are registered with extremely low currents in the range of less than 2 mA at an alternating current frequency in the range of 8-25 Hz. If electrodes are positioned in the F7-F8 area, the patient can close their eyes or keep them open in a dark room. There is no unpleasant pressure in the area of the eyelid or above or below the eye due to electrodes being applied or placed.

If wet electrodes are used and positioned in the F7-F8 area, a very pleasant cooling effect also occurs, which leads to stress relief, reduces skin resistance and also improves the success of the treatment.

With the foregoing in mind, the invention relates to a system for local activation of the human eyes and brain to reorganize neuronal networks for enhancing residual vision and improving blood circulation in existing visual-field defects. In this respect, pulsed current treatment, in particular non-invasive alternating current pulse treatment, is used.

The system consists of an applicator to guide the current flow to the eye and brain and to stimulate blood flow and activation of nerve cells, especially retinal ganglion cells.

The system also includes an impulse generator for generating electrical stimulation signals and a data processing and control unit for providing patient-specific stimulation signal sequences.

The applicator has at least two electrodes that can be applied to the head of the test person in the manner described below.

Replaceable stimulation electrodes are attached to the applicator so that they are positioned to the right and left of the eye in the area of the patient's temple, i.e. in the so-called F7-F8 area.

Furthermore, the applicator has at least one arrangement for the non-invasive determination of blood flow to the skin and brain and for the determination of oxygen saturation, in particular on the basis of fNIR spectroscopy.

An NIR emitter and the at least one associated NIR detector are positioned at a distance from the stimulation electrodes on the patient's head and separately from the stimulation electrodes.

The NIR spectroscopy data, in particular during pauses or at the end of stimulation signal sequences, are recorded in order to determine the duration of sustained, improved blood flow and/or oxygen saturation, so that the data processing and control unit can then be updated via the system according to the invention.

In a further development, the applicator can be individually adapted to the shape of the patient's head. For this purpose, known adjustment means are designed to fix the applicator to the forehead and back region of the patient's head.

In one embodiment of the invention, the applicator is designed as a ring- or crown-shaped structure that can be placed on the head, wherein a nose bridge support and/or an ear support and/or an occipital support is provided.

In addition, a receptacle for electronic components and for weight compensation can be provided in the occipital region. The electronic components can include a battery or an accumulator so that the applicator can be used without external cabling for the power supply.

In one embodiment, the stimulation electrodes are designed as dry or wet pad electrodes.

With a wet pad electrode, an electrolyte can be used as a moisturizing solution to reduce the contact resistance between the respective conductive component in the electrode and the surface of the patient's skin. The lower the contact resistance, the lower the current can be selected for the pulse sequences and there is no unpleasant effect in the sense of a typical tingling sensation when an electric current is felt. Fixation points can be provided on the applicator for attaching, in particular clipping on, further electrodes, which enable current application, in particular alternating current application, also in the area below the patient's eyes, if this is advantageous from a therapeutic point of view.

At least one interface for wired or wireless data transmission is formed on or in the applicator.

In this way, the applicator can exchange information with a higher-level system via a so-called air interface, register the course of the treatment duration and the success of the treatment and make this data available or transmit it to the attending physician for evaluation and further optimization of the treatment.

In a particular embodiment, sound-generating means, in particular designed as earphones or headphones, are provided on the applicator in order to guide the patient through information during treatment, in particular for handling the applicator, and/or to acoustically relax the patient.

For self-sufficient operation, the applicator comprises both the impulse generator, in particular the alternating current impulse generator, and the data-processing and control unit together with the power supply, wherein the treatment sequence and/or the stimulation signal sequences can be realized by a computer program product. In this respect, the applicator therefore has all the technical means necessary for use and treatment. It is therefore no longer necessary to provide a separate impulse generation unit, which is either wired to the patient or has to be carried by the patient, for example as is realized in the known prior art by devices which are fixed to the patient's belt or carried in the patient's pocket.

An electrolyte reservoir for electrode wetting can be provided on or in the applicator. This allows the patient to slightly moisten the electrodes themselves if they begin to dry out. Alternatively, the supply of electrolyte can also be automatically controlled by a suitable sensor via communicating tubes with the electrode holders.

In a further embodiment, the applicator and/or receptacles for holding the electrodes consist of a malleable plastic material. This makes it possible to adapt the applicator individually to certain head shapes, in particular in the forehead, nose or neck area of the patient, thereby providing actual individualization for the patient concerned.

The holders and/or the stimulation electrodes can consist of a conductive plastic material, so that the construction of the applicator is simplified and the power supply or the transmission of the stimulation pulse sequences can be realized via such a conductive material.

It is within the terms of the invention that a pocket or similar holder for replacement electrodes is provided on the applicator. If a replaceable electrode is lost, the treatment can be continued, which is particularly advantageous if the patient to be treated is not in the vicinity of the medical practice that would otherwise care for him.

Especially for stationary or clinical applications, the applicator can be combined with a known EEG electrode cap.

As already indicated, the applicator can have a unit for determining the contact resistance between the stimulation electrodes and the patient's skin surface in order to switch off the impulse generator or reduce the current flow if the contact resistance is abnormal, in particular if it is too high.

The invention will be explained in more detail below with reference to an exemplary embodiment and with the aid of figures, wherein:

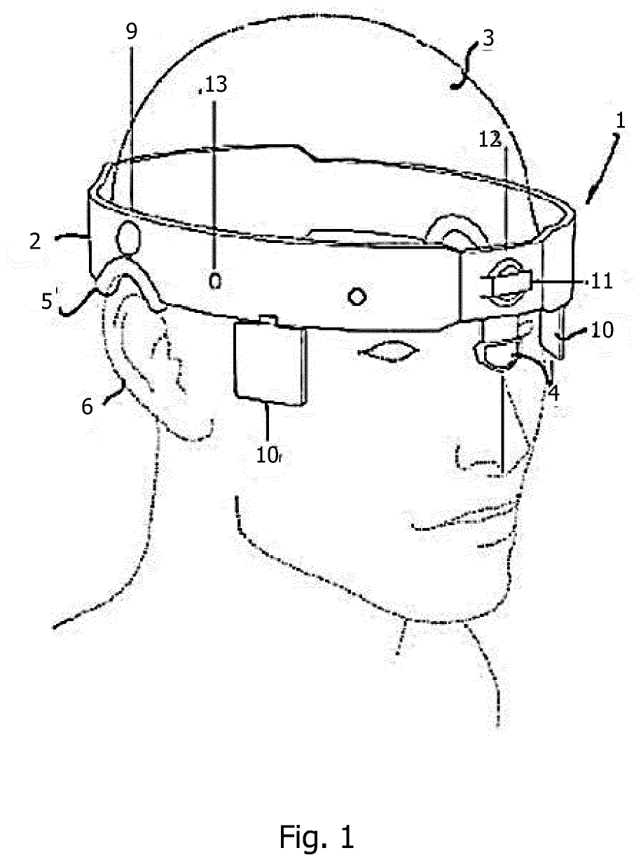

FIG. 1 shows a front view of an exemplary Long COVID applicator attached to the head of a test person; and

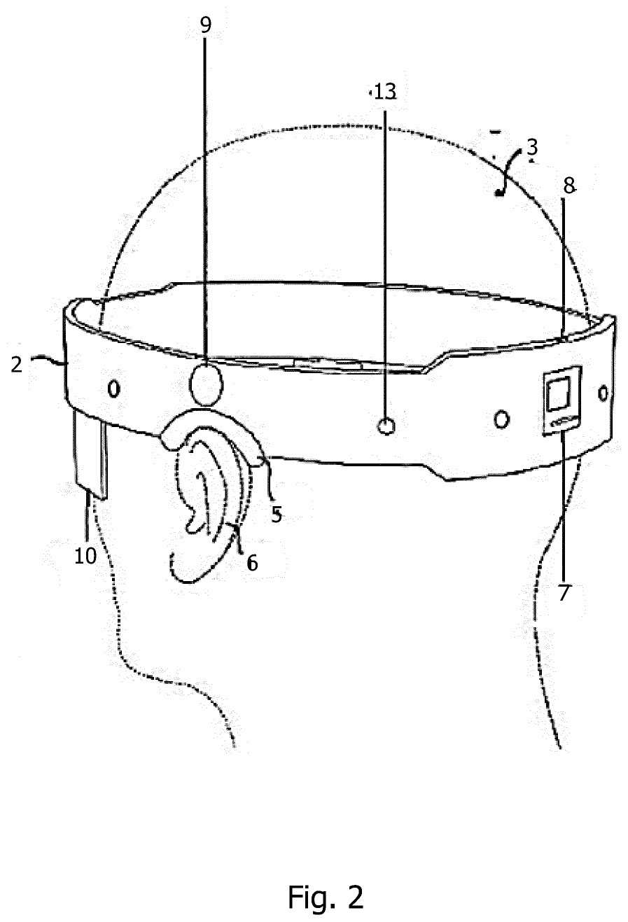

FIG. 2 shows a rear view of the applicator looking at the back of the subject's head.

The applicator 1 according to FIGS. 1 and 2 consists of a base body 2 made of an at least partially elastic material and can be individually adapted to the head shape of the indicated test person 3.

The base body 2 is realized as a ring- or crown-shaped structure that can be placed on the head of the subject 3, with a nasal bridge support 4 that includes an adjustment option for adaptation to anatomical conditions.

Furthermore, the base body 2 is connected to ear supports 5 for the left and right ear 6 of the subject 3.

At the back of the head, the base body 2 is equipped with a holder for electronic components.

On the one hand, this can be a memory unit 7, which comprises stimulation programs. Furthermore, an impulse generator for generating electrical stimulation signals together with a control unit for providing patient-specific stimulation signal sequences can preferably be accommodated or attached in this occipital region.

An interface 8, for example designed as a USB port, can be used for data transmission, but also for charging secondary cells 9 for the purpose of supplying power to the necessary electrical or electronic components.

Such a secondary cell 9 can, for example, be a replaceable lithium battery, which is housed in a corresponding recess in the base body 2.

Stimulation electrodes 10 are interchangeably attached to the base body 2 and are arranged so that they rest on or against the right and left of the eye in the area of the temple of the indicated patient's head.

In the front area of the base body 2 there is an adjustment device 12, which can be provided with a labeling field 11 to create space for a company logo, handling Instructions or also for attaching a user name or user identification,

Sensors 13 can be attached or embedded in the base body 2 in order to carry out a non-invasive determination of the blood flow to the skin and brain and to determine the oxygen saturation. These sensors are located away from the stimulation electrodes 10.

1. System for activating nerve cells in the human eye and brain for enhancing residual vision in the case of existing visual-field defects, for reducing or correcting other visual disorders or cognitive deficits by means of neurostimulation, in particular non-invasive microcurrent application to the head, more particularly non-invasive alternating current pulse stimulation (tACS), consisting of a non-invasive applicator for guiding an energy flow to the eye and brain and for stimulating blood flow and activation of nerve cells, in particular retinal ganglion and brain cells, an impulse generator for generating electrical or magnetic stimulation signals and a data-processing and control unit for providing patient-specific stimulation signal sequences, wherein the applicator comprises, for example, at least two electrodes which can be brought into contact with the head of the test subject and in this respect stimulation electrodes, in particular replaceable electrodes, are fixed to the applicator in such a way that they rest on or against the right and left of the eye in the region of the temple of the patient's head, characterized in that the stimulation signal sequences are repeatedly provided for application until a detectable reduction of an existing vascular dysregulation in the eyes and brain is determined.

2. System according to claim 1, characterized by the use and application for reducing functional long-term complaints due to diseases of viral origin, in particular in connection with the SARS-COV-2 virus.

3. System according to claim 1, characterized in that the applicator can be individually adapted to the shape of the patient's head and adjustment means are provided for this purpose with regard to fixation on the forehead and back region of the patient's head.

4. System according to claim 1, characterized in that the applicator is designed as a ring- or crown-shaped structure which can be placed on the head, wherein a nose bridge support and/or an ear support and/or an occipital support and a receptacle for electronic components and for weight compensation are provided in the occipital region.

5. System according to claim 1, characterized in that fixation points are provided on the applicator for attaching, in particular clipping on, further electrodes, which also enable alternating current to be applied in the area below the patient's eyes.

6. System according to claim 1, characterized in that an interface for wired or wireless data transmission is formed on or in the applicator.

7. System according to claim 1, characterized in that sound generating means, in particular designed as earphones or headphones, are provided on the applicator in order to guide the patient through information and/or acoustically relax the patient during the alternating current pulse treatment, wherein heat, in particular infrared radiant heat, can additionally be supplied to the patient in the application area.

8. System according to claim 1, characterized in that, for self-sufficient operation, the applicator comprises both the alternating current impulse generator and the data-processing and control unit together with the power supply, wherein the treatment sequence and/or the stimulation signal sequences can be realized by a computer program product.

9. System according to claim 1, characterized in that a unit for determining the contact resistance between the stimulation electrodes and the patient's skin surface is designed to switch off the impulse generator in the event of abnormal, in particular excessive, resistances.

10. System according to claim 1, characterized in that the applicator comprises at least one arrangement for non-invasive determination of blood flow to the skin and brain and for determination of oxygen saturation, in particular based on fNIR spectroscopy, wherein the NIR emitter and the at least one associated NIR detector can be positioned in remote position from the stimulation electrodes on the patient's head and separately from the stimulation electrodes, and the NIR spectroscopy data are recorded, in particular during pauses or at the end of stimulation signal sequences, in order to determine the duration of a sustained, improved blood flow and/or oxygen saturation, so that an updated operation of the data processing and control unit can be realized thereafter.

11. System according to claim 1, characterized in that the position of the stimulation electrodes corresponds to an F7-F8 arrangement according to the nomenclature of the EEG 10-20 system.

12. System according to claim 1, characterized in that in the case of preferred microcurrent application, the current intensity of the stimulation signals is increased stepwise according to a predeterminable regime, in particular as a function of visual impressions, such as flashes of light or the like, and/or the stimulation parameters are adjusted or fixed as a function of the dilatation capacity of the blood vessels in the eye.
